(19) United States
(12) Patent Application Publication
(10) Pub. No.: US 2025/0065037 A1
(43) Pub. Date: Feb. 27, 2025

(54) PUMP INTERCONNECTIVITY FOR PAIN MEDICATION THERAPIES

(71) Applicants: BAXTER INTERNATIONAL INC., Deerfield, IL (US); BAXTER HEALTHCARE SA, Glattpark (Opfikon) (CH)

(72) Inventors: Rajesh SWARNKAR, Bangalore, Karnataka (IN); Prasad Deepak DIGASKAR, Bangalore, Karnataka (IN); Saman Rais Alam KHAN, Bangalore, Karnataka (IN)

(21) Appl. No.: 18/725,277

(22) PCT Filed: Dec. 28, 2022
(86) PCT No.: PCT/US2022/082457

§ 371 Date: Jun. 28, 2024

(30) Foreign Application Priority Data
Dec. 30, 2021 (IN) 202141061803

(51) Int. Cl. A61M 5/142; A61M 5/14

(52) U.S. Cl. CPC A61M 5/142; A61M 2005/1405; A61M 2005/14208

(57) ABSTRACT

A system, apparatuses, and methods are disclosed that provide pump interconnectivity for pain medication therapies. In an example embodiment, a system includes a patient-controlled analgesia (“PCA”) pump, an infusion pump, and a hub device configured to connect to the PCA pump and the infusion pump. The hub device is configured to determine the PCA pump and the infusion pump are both communicatively connected to the hub device, enable the PCA pump and the infusion pump to communicate with each other for a pain medication therapy, and enable the infusion pump to deliver the fluid to the patient when the infusion pump detects a period between periodic PCA pump boluses.

### BACKGROUND

Patient-controlled analgesia (“PCA”) pumps are commonly used for administering pain medication intravenously. PCA pumps are programmed to deliver a bolus of pain medication continuously or intermittently at programmed times, such as every thirty minutes. PCA pumps are often connected to a patient handheld controller. If a patient's pain becomes too great to tolerate, the patient is permitted to activate a button or other input on the controller that causes the PCA pump to administer another bolus. To keep a patient from overdosing, PCA pumps are programmed to prevent a patient from actuating the button or other input too many times during a time duration.

One known issue with the intermittent mode of PCA pumps is that there is no pain medication flow between the periodic boluses. There is a risk that a patient's vein can become occluded or otherwise collapse between the periodic boluses. To prevent a vain from occluding, a syringe pump or large volume pump (“LVP”) may also be connected to the patient at the same intravenous access point (via a T-connector) to provide a slow drip or release of saline or similar fluid.

The PCA and LCP/syringe pumps are typically separate devices. As such, a clinician is required to program each pump separately. This means that a clinician has to program both pumps with similar start times and ensure the fluid delivery is compatible. Issues can occur when the pumps are not programmed together correctly. Further, there is no communication between the pumps such that one pump may administer fluid/medication normally even when the other pump experiences an issue or is removed altogether.

A need accordingly exists for pump interconnectivity for pain medication therapies.

### SUMMARY

The present disclosure sets forth systems, apparatuses, and methods for pump interconnectivity during pain medication therapies. The systems, apparatuses, and methods include at least two pumps. A first pump is a PCA pump that is configured to continuously or intermediately deliver a pain medication to a patient. A second pump is an infusion pump (e.g., a linear peristaltic pump, a large volume pump (“LVP”), an ambulatory pump, or a multi-channel pump, etc.) that is configured to deliver a fluid such as saline to the patient. The first pump is connected to the second pump via a hub device that supports interconnectivity via a controller area network (“CAN”) connection, an Ethernet connection, a serial connection, and/or a universal serial bus (‘USB”). In other embodiments, the first pump is connected to the second pump via a wireless connection such as Bluetooth®.

The first and second pumps are interconnected such that status messages and/or event messages are shared directly via the hub device without needing an external connection to a hospital information system. The first and second pumps are configured to transmit status messages between themselves including event messages, alert messages, alarm messages, etc. The transmission of the messages provides information to each pump regarding respective infusion and PCA therapies. The PCA pump is configured to, for example, pause medication or fluid delivery when the infusion pump generates an alert or alarm that necessitates stoppage, such as detection of a line occlusion, leak, adverse patient condition, etc. Further, the device interconnectivity is configured to generate an alert at one or both of the pumps when the other pump is disconnected from the hub device.

Aspects of the subject matter described herein may be useful alone or in combination with one or more other aspect described herein. Without limiting the foregoing description, in a first aspect of the present disclosure, a system for intravenously administering pain medication includes a patient-controlled analgesia (“PCA”) pump configured to deliver a pain medication to a patient and an infusion pump configured to deliver a fluid to the patient. The system also includes a hub device configured to receive the PCA pump and the infusion pump. The hub device includes a communication interface configured to communicatively couple to each of the PCA pump and the infusion pump, and a processor communicatively coupled to the communication interface. The processor is configured to determine the PCA pump and the infusion pump are both communicatively connected to the communication interface. The processor enables the PCA pump and the infusion pump to communicate with each other for a pain medication therapy. The processor also enables the infusion pump to deliver the fluid to the patient when the infusion pump detects a period between periodic PCA pump boluses.

In a second aspect of the present disclosure, which may be combined with any other aspect listed herein, the infusion pump is configured to detect the period between periodic PCA pump boluses using an event message received from the PCA pump, the event message indicative that the PCA pump is to administer or has administered a bolus of the pain medication.

In a third aspect of the present disclosure, which may be combined with any other aspect listed herein, the PCA pump generates the event message after reaching a programmed scheduled bolus or receiving an input from a patient controller.

In a fourth aspect of the present disclosure, which may be combined with any other aspect listed herein, the infusion pump is configured to detect the period between periodic PCA pump boluses after receiving a bolus schedule that is indicative of times that the PCA pump is to administer a bolus of the pain medication.

In a fifth aspect of the present disclosure, which may be combined with any other aspect listed herein, the communication interface is configured to provide at least one of a controller area network (“CAN”) connection, an Ethernet connection, a serial connection, or a universal serial bus (‘USB”) connection between the PCA pump and the infusion pump.

In a sixth aspect of the present disclosure, which may be combined with any other aspect listed herein, the fluid is saline.

In a seventh aspect of the present disclosure, which may be combined with any other aspect listed herein, the infusion pump is configured to detect its removal from the hub device by detecting a lost connection with the communication interface, and cause an alert to be generated that is indicative of a disconnection from the pain medication therapy.

In an eighth aspect of the present disclosure, which may be combined with any other aspect listed herein, the infusion pump is configured to detect a removal of the PCA pump from the hub device based on a lost connection with the communication interface or receive a message indicative that the PCA pump has been removed from the hub device, and cause an alert to be generated that is indicative of a disconnection of the PCA pump.

In a ninth aspect of the present disclosure, which may be combined with any other aspect listed herein, the PCA pump is configured to detect its removal from the hub device by detecting a lost connection with the communication interface, and cause an alert to be generated that is indicative of a disconnection from the pain medication therapy.

In a tenth aspect of the present disclosure, which may be combined with any other aspect listed herein, the PCA pump is configured to detect a removal of the infusion pump from the hub device based on a lost connection with the communication interface or receive a message indicative that the infusion pump has been removed from the hub device, and cause an alert to be generated that is indicative of a disconnection of the infusion pump.

In an eleventh aspect of the present disclosure, which may be combined with any other aspect listed herein, the system further includes an intravenous (“IV”) y-connector with an outlet end connected to the patient, a first inlet end connected to the PCA pump, and a second inlet end connected to the infusion pump.

In a twelfth aspect of the present disclosure, which may be combined with any other aspect listed herein, the infusion pump includes at least one of a syringe pump, a linear peristaltic pump, a large volume pump (“LVP”), an ambulatory pump, or a multi-channel pump.

In a thirteenth aspect of the present disclosure, which may be combined with any other aspect listed herein, the infusion pump is configured to detect that the PCA pump has started the pain medication therapy, and cause an alert to be generated that is indicative that the infusion pump has not yet been started for the pain medication therapy.

In a fourteenth aspect of the present disclosure, which may be combined with any other aspect listed herein, the PCA pump is configured to detect that the infusion pump has started the pain medication therapy, and cause an alert to be generated that is indicative that the PCA pump has not yet been started for the pain medication therapy.

In a fifteenth aspect of the present disclosure, which may be combined with any other aspect listed herein, the infusion pump is configured to detect that a PCA pump has not yet been connected to the hub device, and cause an alert to be generated that is indicative that the PCA pump should be connected to the hub device for the pain medication therapy.

In a sixteenth aspect of the present disclosure, which may be combined with any other aspect listed herein, the PCA pump is configured to detect that an infusion pump has not yet been connected to the hub device, and cause an alert to be generated that is indicative that the infusion pump should be connected to the hub device for the pain medication therapy.

In a seventeenth aspect of the present disclosure, which may be combined with any other aspect listed herein, a hub apparatus is configured to connect to (i) a patient-controlled analgesia (“PCA”) pump for delivering a pain medication to a patient, and (ii) an infusion pump for delivering a fluid to the patient. The hub apparatus includes a communication interface configured to communicatively couple to each of the PCA pump and the infusion pump, and a processor communicatively coupled to the communication interface. The processor is configured to determine the PCA pump and the infusion pump are both communicatively connected to the communication interface, enable the PCA pump and the infusion pump to communicate with each other for a pain medication therapy, and enable the infusion pump to deliver the fluid to the patient when the infusion pump detects a period between periodic PCA pump boluses.

In an eighteenth aspect of the present disclosure, which may be combined with any other aspect listed herein, the communication interface is configured to provide at least one of a controller area network (“CAN”) connection, an Ethernet connection, a serial connection, or a universal serial bus (‘USB”) connection between the PCA pump and the infusion pump.

In a nineteenth aspect of the present disclosure, which may be combined with any other aspect listed herein, the hub apparatus further includes a network connectivity stage configured to house the communication interface and the processor, and a pump stage configured to connect to the PCA pump and the infusion pump.

In a twentieth aspect of the present disclosure, which may be combined with any other aspect listed herein, the pump stage is removably connected to the network connectivity stage and when connected, the pump stage is communicatively coupled to the network connectivity stage.

In a twenty-first aspect of the present disclosure, any of the structure and functionality illustrated and described in connection with FIGS. 1 to 6 may be used in combination with any of the structure and functionality illustrated and described in connection with any of the other of FIGS. 1 to 6 and with any one or more of the preceding aspects.

In light of the aspects above and the disclosure herein, it is accordingly an advantage of the present disclosure to provide a system that provides for network-less connectivity between an infusion pump and a PCA pump via a hub device.

It is another advantage of the present disclosure to provide a system that provides PCA and infusion pump interconnectivity to enable a slow delivery of saline to keep a patient's vein open between boluses of pain mediation delivered by the PCA pump.

Additional features and advantages are described in, and will be apparent from, the following Detailed Description and the Figures. The features and advantages described herein are not all-inclusive and, in particular, many additional features and advantages will be apparent to one of ordinary skill in the art in view of the figures and description. Also, any particular embodiment does not have to have all of the advantages listed herein and it is expressly contemplated to claim individual advantageous embodiments separately. Moreover, it should be noted that the language used in the specification has been selected principally for readability and instructional purposes, and not to limit the scope of the inventive subject matter.

### BRIEF DESCRIPTION OF THE FIGURES

FIG. 1 shows a diagram of a medical system configured for the example methods, apparatuses, and systems described herein, according to an example embodiment of the present disclosure.

FIG. 2 shows a diagram of an example infusion pump comprising the Baxter® SIGMA Spectrum pump, which may be included within the medical system of FIG. 1, according to an example embodiment of the present disclosure.

FIG. 3 is a diagram of a hub device of the medical system of FIG. 1, according to an example embodiment of the present disclosure.

FIG. 4 is a diagram showing the interconnectivity of a PCA pump and an infusion pump of the medical system of FIG. 1, according to an example embodiment of the present disclosure.

FIG. 5 is a diagram of the hub device connected to the infusion pump and the PCA pump of FIGS. 1 and 4, according to an example embodiment of the present disclosure.

FIG. 6 is a flow diagram of an example procedure for performing a pain medication therapy using the example hub device, according to an example embodiment of the present disclosure.

### DETAILED DESCRIPTION

The present disclosure relates in general to methods, systems, and apparatuses for pump interconnectivity during pain medication therapies. The methods, systems, and apparatuses are configured to coordinate communication between at least one PCA pump and at least one infusion pump. The PCA pump and the infusion pump are fluidly coupled to a patient via an intravenous y-connector. As described herein, the infusion pump is configured to provide a slow delivery of saline or similar solution between boluses of pain medication provided by the PCA pump. The methods, systems, and apparatuses described herein are configured to use the infusion pump to keep a patient's vein open between deliveries of the pain medication by the PCA pump. The interconnectivity between the PCA pump and the infusion pump ensures the deliveries of saline and pain medication are coordinated and ensures one or more alerts/alarms are generated when one of the pumps is deactivated or otherwise moved.

The methods, systems, and apparatuses provide pump connectivity using a device hub. As described herein, a device hub provides connectivity between two or more pumps via one or more of an Ethernet connection, a serial connection, a USB connection, and/or a CAN connection. The pump connectivity provided by the hub device is local such that a connection to a hospital information system is not needed. As such, the methods, systems, and apparatuses may be used in rural and other areas where an Internet and/or a cellular connection may not be readily available.

Reference is made herein to medical device data that is communicated between the PCA and infusion pumps. The medical device data may be transmitted/broadcasted in one or more messages. As disclosed herein, medical device data includes device operating parameters, treatment/therapy progress, alarms/alerts, events, diagnostic information, etc. For an infusion pump, the medical device data may include an infusion rate, a dose, a total volume infused, a time remaining for a treatment/therapy, a medication concentration, rate change, a volume remaining within a medication container, a medication name, a patient identifier, titration information, bolus information, a care area identifier, a timestamp when the data was generated, an alarm condition, an alert condition, an event, etc. The medical device data may also include event information, such as when a pump begins or is about to begin to deliver fluid to a patient, when a pump is stopped or paused, and/or when an error condition is detected.

### Medical Environment Embodiment

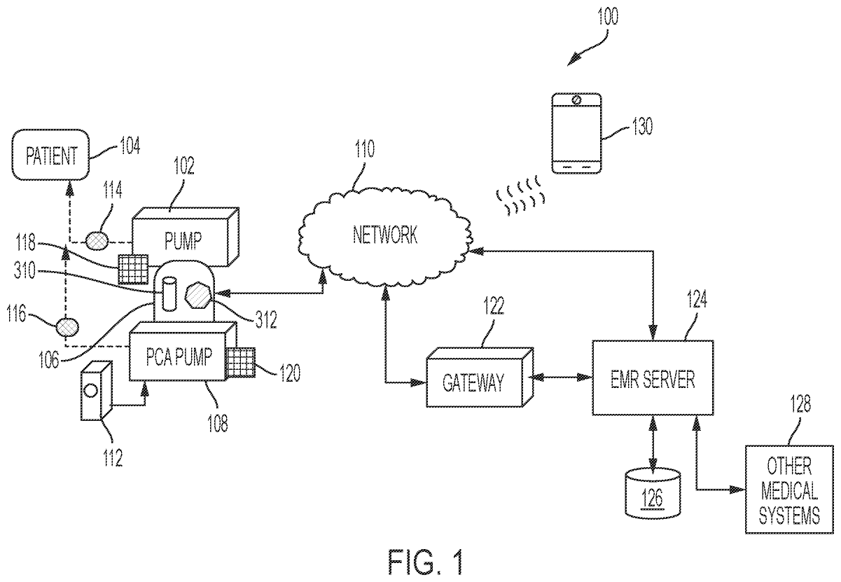

FIG. 1 shows a diagram of a medical system 100 configured for the example methods, apparatuses, and systems described herein, according to an example embodiment of the present disclosure. The example system 100 includes one or more medical device such as, for example, a first pump 102. The example first pump 102 may include any pump capable of delivering an intravenous therapy to a patient 104 via one or more intravenous (“IV”) line sets. Examples include a syringe pump, a linear peristaltic pump, a large volume pump (“LVP”), an ambulatory pump, multi-channel pump, etc. A syringe pump uses a motor connected to a drive arm to actuate a plunger within a syringe. A linear peristaltic pump uses a rotor to compress part of a tube while rotating. Oftentimes, one or more rollers of the rotor contact the tube for half a rotation. The compressed rotation causes a defined amount of fluid to pass through the tube. LVPs typically use one or more fingers or arms to compress a portion of intravenous therapy (“IV”) tube. The timing of the finger actuation on the tube causes constant or near constant movement of a fluid through the tube.

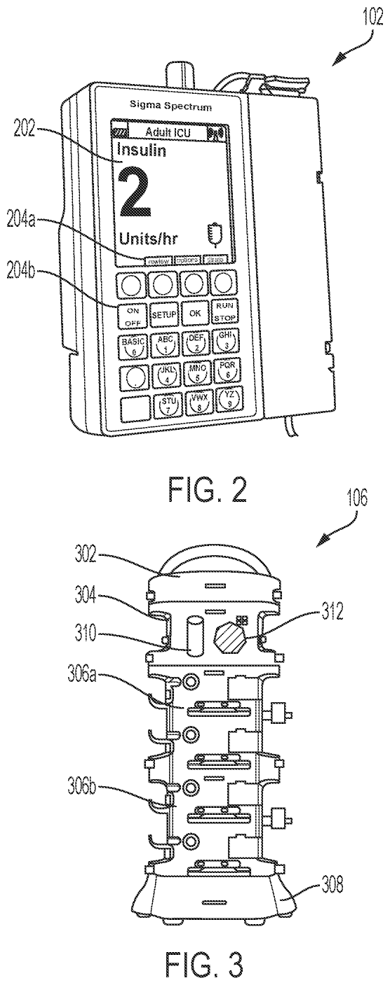

FIG. 2 shows a diagram of an example infusion pump 102, according to an example embodiment of the present disclosure. The illustrated infusion pump 102 is the Baxter® SIGMA Spectrum™ pump. The illustrated infusion pump 102 includes a display 202 with interfaces 204a and 204b to enable a clinician to specify or program an infusion therapy.

Other examples of infusion pumps that may be included in the medical system 100 of FIG. 1 include a linear volume parenteral pump described in U.S. Publication No. 2013/0336814, a syringe pump described in U.S. Publication No. 2015/0157791, an ambulatory infusion pump described in U.S. Pat. No. 7,059,840, an infusion pump described in U.S. Pat. No. 5,395,320,and an infusion pump described in U.S. Pat. No. 5,764,034, the entirety of each are incorporated herein by reference.

The example infusion pump 102 is connected to a hub device 106. Additionally in the illustrated example, the hub device 106 includes a PCA (e.g., a second) pump 108. FIG. 3 is a diagram of the hub device 106, according to an example embodiment of the present disclosure. The hub device 106 includes a handle stage 302, a network connectivity stage 304, at least one pump stage 306 (two pump stages 306a and 306b are shown), and a base stage 308 (optional).

The example handle stage 302 is configured to enable the hub device 106 to be carried by an operator. The hub device 106 may be mounted to a pole or mounted to a patient's bed. The example network connectivity stage 304 is connected to the handle stage 302 and includes a communication interface 310 and a processor 312. The communication interface 310 is configured to detect how many pump stages 306 are connected in series. For example, as many as six pump stages 306 may be connected. The communication interface 310 is also configured to provide connectivity between pumps provided in the pump stages 306. Further, the communication interface 310 is configured to provide connectivity between the pumps in the pump stages 306 and a network 110, shown in FIG. 1. The communication interface 310 may be configured to enable the pumps to communicate with each other via USB connections, Ethernet connections, CAN connections, and/or serial connections. In other embodiments, the first pump 102 is connected to the PCA pump 108 via a wireless connection such as Bluetooth®.

The example processor 312 of the hub device 106 is configured to provide routing for communications among pumps within the pump stages 306 via the communication interface 310. During an initial registration routine, the processor 312 detects when pump stages 306 are connected to the hub device 106 and/or when pumps are connected to one of the pump stages 306. The processor 312 is configured to assign a channel to each pump. As such, messages transmitted from each pump of the hub device 106 are routed/broadcast to other pumps of the hub device via the serial, USB, CAN, or Ethernet connection.

In some embodiments, an operator may use the interface 202 of one of the pumps to associate another one of the pumps connected to the hub device 106 to a same patient and/or treatment. In these embodiments, the processor 312 transmits a list of the connected pumps (with an ID or other identifier) to the pump for display and selection by the operator. The association process may be programmed into a pump as part of a setup routine for certain treatments.

The base stage 308 of FIG. 3 is configured to provide support for the hub device 106. In some embodiments, the base stage 308 may include a battery and connection to a power source. In other embodiments, the network connectivity stage 304 includes the battery and/or the power source connectivity. The base stage 308 may be optional.

Returning to FIG. 1, the hub device 106 is connected to the first pump 102 and the PCA pump 108. In other embodiments, the hub device 106 may include additional first pumps 102 and/or additional PCA pumps 108. For example, some treatments may include two PCA pumps 108 and one first pump 102 connected to the hub device 106.

The PCA pump 108 is connected via a wire to a handheld controller 112. While the PCA pump 108 may be programmed to intermittently or continuously provide pain medication, the controller 112 includes a button that when pressed causes the PCA pump 108 to administer a bolus of pain medication (or temporarily increase a rate of delivery).

As shown in FIG. 1, the first pump 102 is configured to deliver a first fluid 114 to a patient. The first fluid 114 may include saline or any other fluid/medication for administration to the patient 104. The PCA pump 108 is configured to deliver a second fluid 116 to the patient 104. The second fluid 116 may include a pain medication, such as a fluid-based opioid.

The first pump 102 includes a processor 118 configured to execute machine-readable instructions stored in a memory device. Execution of the machine-readable instructions by the processor 118 causes the first pump 102 to perform the operations described herein. Additionally, the second pump 108 includes a processor 120 configured to execute machine-readable instructions stored in a memory device. Execution of the machine-readable instructions by the processor 120 causes the second pump 108 to perform the operations described herein.

In some embodiments, the example hub device 106 is communicatively coupled to a gateway 122 via the network 110. The example gateway 122 is configured to receive infusion pump data and/or PCA data (e.g., medical device data) from the infusion pump 102 and the PCA pump 108, and route the data to an EMR server 124. In some embodiments, the gateway 122 is configured to convert the data from, for example, EXTCOM message(s) to HL7 message(s). In yet other embodiments, the network 110 and the gateway 122 are omitted from the system 100.

The example gateway 122 may also be configured to transmit operating parameters or prescription parameters to the first pump 102 and/or the PCA pump 108. For example, the gateway 122 may transmit an electronic prescription (or software update) to the first pump 102 at a predetermined time and/or when the first pump 102 is available to accept the prescription. In other instances, the first pump 102 and/or the PCA pump 108 may be configured to periodically poll the gateway 122 to determine when an electronic prescription (or software update) is awaiting to be downloaded to the pump. The first pump 102 and/or the PCA pump 108 may include a memory storing one or more drug libraries that include particular program parameter limits based on care area, dose change, rate of change, drug type, concentration, patient age, patient weight, etc. The limits are configured to ensure that a received prescription or entered infusion therapy is within acceptable ranges and/or limits decided by a medical facility, doctor, or clinician.

The first pump 102 is configured to perform an infusion therapy or treatment for the patient 104, which includes infusing one or more solutions 114 or medications into the patient. The first pump 102 operates according to an infusion prescription entered by a clinician at a user interface of the pump (e.g., the interface 204 of FIG. 2) or received via the infusion gateway 122. The first pump 102 may compare the prescription to the drug library and provide any alerts or alarms when a parameter of the prescription exceeds a soft or hard limit. The first pump 102 is configured to monitor the progress of the therapy and periodically transmit infusion therapy progress data (e.g., medical device data) to the gateway 122. The therapy progress data, as disclosed herein, may include, for example, an infusion rate, a dose, a total volume infused, a time remaining for the therapy, a medication concentration, rate change, a volume remaining within a medication container, a medication name, a patient identifier, titration information, bolus information, a care area identifier, a timestamp when the data was generated, an alarm condition, an alert condition, an event, etc. The first pump 102 may transmit the data continuously, periodically (e.g., every 30 seconds, 1 minute, etc.), or upon request by the gateway 122.

The PCA pump 108 is configured to perform a pain medication therapy or treatment for the patient 104. Similar to the first pump 102, the PCA pump 108 is configured to receive a prescription from the gateway 122 or a user interface. Further, similar to the first pump 102, the PCA pump 108 is configured to transmit therapy progress data to the gateway 122.

As disclosed herein, the hub device 106 provides direct connectivity to the network 110. The network connectivity stage 304 of the hub device 106 receives the therapy progress data from the pumps 102/108 via a serial, Ethernet, CAN, or USB connection. The network connectivity stage 304 then converts the therapy progress data to a protocol for transmission via Ethernet to the gateway 122 via the network 110. If the pumps 102/108 are connected via an Ethernet to the network connectivity stage 304, the network connectivity stage 304 may be configured as a network router. The network connectivity stage 304 performs similar operations to convert prescriptions and other external messages into the specified communication protocol for transmission to the specified pump 102/108.

The gateway 122 of FIG. 1 includes a server, processor, computer, etc. configured to communicate with the hub device 106. The gateway 122 may include, for example, the Baxter® IQ Enterprise® gateway. In some embodiments, the gateway 122 may be communicatively coupled to more than one hub device. The gateway 122 is configured to provide bi-directional communication with the pumps 102/108 for the wired/wireless secure transfer of drug libraries, infusion prescriptions, and therapy progress data. The gateway 122 may also be configured to integrate with the EMR server 124 or other hospital system to facilitate the transmission of the infusion therapy progress data from the pumps 102/108 to, for example, a hospital electronic medical record (“EMR”) related to the patient 104.

The example EMR server 124 is communicatively coupled to an EMR database 126 for storing EMR records. The EMR database 126 may also be configured to store infusion pump data that is not associated to a particular patient and/or medication order. The EMR server 124 is also communicatively coupled to a pharmacy server, which is configured to create and/or transmit medication orders corresponding to, for example, prepared fluids 114/116. A medication order includes an electronic record or entry, which identifies a patient (e.g., a patient identifier) and infusion parameters for administration. The medication order is assigned a unique identifier. In some embodiments, the medication order may be printed on a label attached to a medication container that is fluidly coupled to one of the pumps 102/108. The medication order itself associates a patient identifier with a medication identifier. The example EMR server 124 is configured to use the patient identifier in the medication order to store or otherwise associate the medication order with a patient's EMR.

In some examples, the gateway 122 and/or the EMR server 124 includes a memory storing machine-readable code or instructions, that when executed by a processor, cause the gateway 122 and/or the EMR server 124 to perform the operations described herein. This includes operating according to a predefined application, routine, or algorithm.

While the EMR database 126 is shown as being connected directly to the EMR server 124, in other examples they may be connected via the network 110. The example network 110 may include any wired or wireless connection (e.g., an Ethernet network, LAN, WLAN, etc.). The example EMR database 126 may be stored in any volatile or non-volatile memory device including RAM, ROM, flash memory, magnetic or optical disks, optical memory, or other storage media. The database 126 may be structured as a relational database or a graph database. When the database 126 includes a graph database, patients, medication orders, medication device data, and identifiers may be provided by separate nodes.

The example system 100 of FIG. 1 may also include other medical systems 128, such as a hospital information system (“HIS”). An HIS 128 may include one or more servers for analyzing medical data or receiving medical data from other portions of the medical system 100. The HIS 128 may include or be communicatively coupled to a laboratory information system, pharmacy system, a policy/procedure management system, or a continuous quality improvement (“CQI”) system. The laboratory system is configured to generate medical data based on analysis of patient biological samples. The policy/procedure management system is configured to manage drug libraries and/or thresholds for alarms/alerts. The CQI system is configured to generate statistical and/or analytical reports based on, for example, therapy progress data from one or more patients. The HIS 128 may also include or be connected to one or more monitors configured to display at least a portion of the therapy progress data.

In some embodiments, the first pump 102 and/or the PCA pump 108 may also be communicatively coupled to one or more physiological sensors. For example, the first pump 102 may be connected to a pulse oximetry sensor, a blood pressure cuff, an access disconnection device, a temperature sensor, a heat rate sensor, and/or a weight scale. The first pump 102 may be configured to integrate or otherwise include data from the pulse oximetry sensor into the therapy progress data or, alternatively, transmit the pulse oximetry data separately to the gateway 122.

The example system 100 of FIG. 1 also includes a clinician device 130 (e.g., a smartphone, tablet computer, laptop computer, workstation, etc.) that enables a clinician to view patient data stored at the EMR database 126. The clinician device 130 may include one or more interfaces or applications for reading and/or writing data stored at the database 126. The clinician device 130 is configured to enable a clinician to view, for example, medication orders for a patient, a patient's EMR, and/or medical device data associated with a patient. In some embodiments, the clinician device 130 may be configured to connect to the hub device 106 for associating the pumps 102/108 with a same treatment for the patient 104.

### Connectivity Embodiment

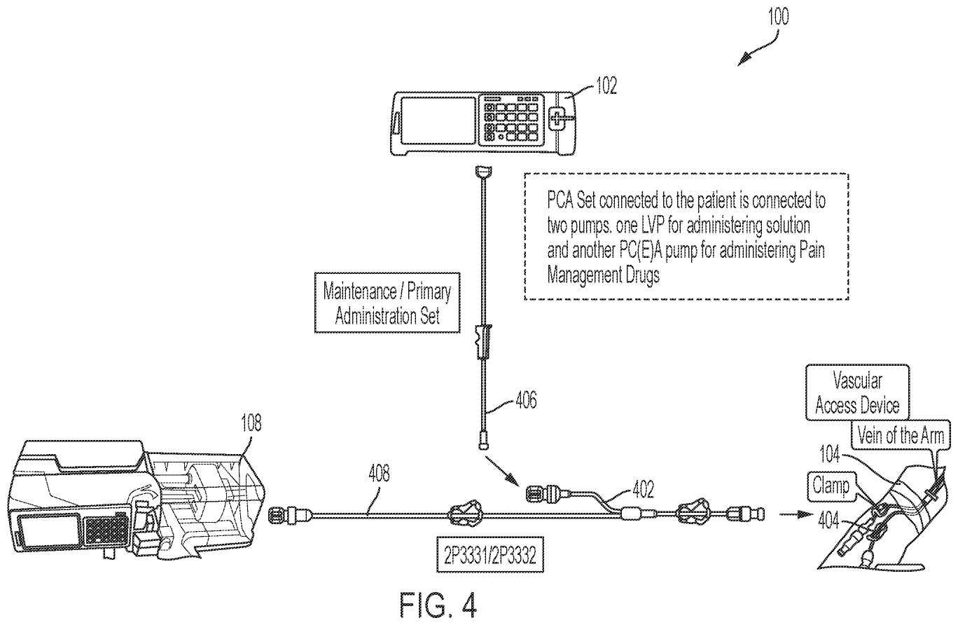

As discussed above, the hub device 106 is configured to enable the PCA pump 108 and the infusion (first) pump 102 to create an interconnectivity association for one or more pain medication therapies. FIG. 4 is a diagram showing the interconnectivity of the PCA pump 108 and the infusion pump 102 of the medical system 100, according to an example embodiment of the present disclosure. In the illustrated example, an intravenous y-connector 402 includes an outlet end that is fluidly coupled to a vascular access device 404 that is inserted into a vein of a patient 104. The y-connector 402 also includes a first inlet end that is fluidly coupled to a first IV line 406, which is routed to the first pump 102 for providing the first fluid 114. The y-connector 402 further includes a second inlet end that is fluidly coupled to a second IV line 408 that is routed to the PCA pump 108 for providing the second fluid 116. The y-connector 402 enables both the first and second fluids 114 and 116 to be administered to the patient 104 via the same vascular access device 404. In other embodiments, an epidural connector may be used in place of the vascular access device 404.

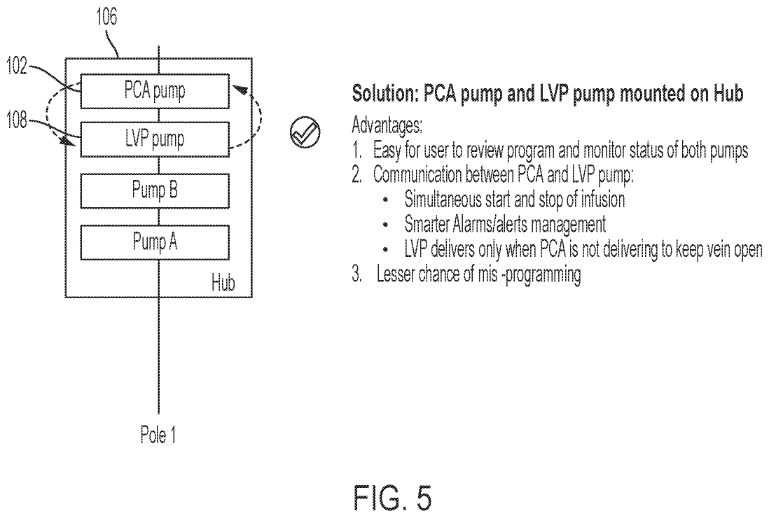

FIG. 5 is a diagram of the hub device 106 connected to the first pump 102 and the PCA pump 108 of FIGS. 1 and 4, according to an example embodiment of the present disclosure. FIG. 5 also shows that the hub device 106 may include additional pumps. The hub device 106 enables the first pump 102 and the PCA pump 108, which are fluidly coupled to the same patient, to associate or otherwise communicate with each other. As described above, the hub device 106 may route messages transmitted from one pump to all other pumps that are connected to the hub device 106. Alternatively, an operator may use an interface of the first pump 102 and/or the PCA pump 108 to create an association. In some embodiments, the hub device 106 provides a list of connected pumps that are selectable from the user interface to create an association. Additionally or alternatively, an operator may use an application on the clinician device 130 to access the hub device 106 and/or the pumps 102/108 to create the association.

The created association or communicative coupling enables the pumps 102/108 to start their respective treatments simultaneously. For example, the first pump 102 may receive an event message that the PCA pump 108 has begun a PCA treatment. When the first pump 102 does not begin its treatment within a threshold time duration (e.g., 60 seconds, 2 minutes, 5 minutes, etc.), the first pump 102 is configured to generate an alert indicative that the infusion therapy should also begin. Additionally or alternatively, the PCA pump 108, after starting, may detect that an event message has not been received from the first pump 102 that is indicative of its start. In response, the PCA pump 108 generates an alert indicative that the first pump 102 should be started. In this instance, the PCA pump 108 may include a prescription that specifies an infusion should also occur. The PCA pump 108 uses this flag and the detection of an infusion start event message to confirm the therapy is operating as prescribed.

In some further embodiments, the prescription may include programming parameters for the first pump 102. In these embodiments, the PCA pump 108 is configured to transmit a message to the first pump 102 with the parameters to program an infusion treatment. In some instances, the first pump 102 receives the message, programs the prescribed treatment, and prompts an operator to confirm the treatment can begin. It should be appreciated that the above-described embodiments may be reversed where the first pump 102 is started first and/or receives a prescription including PCA parameters for the PCA pump 108.

During treatment, the infusion (first) pump 102 receives event messages and/or other medical device data from the PCA pump 108. Further, the PCA pump 108 receives event message and/or other medical device data from the infusion pump 102. The infusion pump 102 is configured to determine when the PCA pump 108 intermittently or periodically administers boluses of pain medication. In other words, the infusion pump 102 determines periods when the bolus is administered. The infusion pump 102 may receive an event message from the PCA pump 108 that is indicative that the PCA pump 108 is about to administer a bolus or has administered a bolus. In response to this event message, the infusion pump 102 is configured to pause delivery of its fluid until the bolus has ended. The event message may specify a duration for the bolus. Alternatively, the pause may be temporary to correspond to a typical, known time to deliver a bolus. The event messages are transmitted during normally scheduled boluses and after a patient has requested a bolus via the controller 112 (when the patient is permitted to receive an extra bolus). For continuous basal deliveries, the PCA pump 108 transmits one or more messages indicative of a duration of the delivery and/or a message indicative that a delivery has stopped. In response, the infusion pump 102 pauses delivery until the continuous basal delivery has ended or paused. In some instances, the infusion pump 102 may delivery saline at a slow rate that is increased when the PCA pump 108 stops or pauses.

In other instances, the PCA pump 108 may transmit a message to the infusion pump 102 with an administration schedule. In these other instances, the infusion pump 102 is configured to deliver its fluid at times when the PCA pump 108 is not scheduled to deliver a bolus of pain medication. The PCA pump 108 may still transmit event messages for patient-controlled boluses.

During a pain medication treatment, the pumps 102/108 are configured to detect disconnection from the hub device 106 and generate a corresponding alert or alarm. The pump 102/108 that is removed from the hub device 106 may detect a lost connection with the hub device 106 by, for example, detecting a loss of power from the hub's power source or the failed transmission of medical device data including event messages.

In an example, the first pump 102 is configured to detect its removal from the hub device 106 by detecting a lost connection with the communication interface 310 and cause an alert to be generated on its use interface that is indicative of a disconnection from the pain medication therapy. Similarly, the PCA pump 108 is configured to detect its removal from the hub device 106 by detecting a lost connection with the communication interface 310 and cause an alert to be generated that is indicative of a disconnection from the pain medication therapy. The first pump 102 may also be configured to detect a removal of the PCA pump 108 from the hub device 106. The removal of the PCA pump 108 may be detected based on a lost connection with the communication interface 310 or the PCA pump 108 or the first pump 102 may receive a message indicative that the PCA pump 108 has been removed from the hub device 106. In response to the lost connection, the first pump 102 causes an alert to be generated that is indicative of a disconnection of the PCA pump 108. Further, the PCA pump 108 is configured to detect a removal of the first pump 102 from the hub device 106 based on a lost connection with the communication interface 310 or receive a message indicative that the first pump 102 has been removed from the hub device. Based on this detected lost connection, the PCA pump 108 causes an alert to be generated that is indicative of a disconnection of the first pump 102.

The interconnectivity between the pumps 102/108 may also ensure that both pumps are stopped at the same time when an issue is detected with the vascular access device 404 or the y-connector 402. For example, an operator may stop the PCA pump 108 when an issue with the vascular access device 404 is detected. In addition to stopping, the PCA pump 108 transmits an event message to the first pump 102 that is indicative of its stoppage. In response, the first pump 102 may prompt an operator to pause its delivery of fluid by displaying an alert on a user interface. Such a configuration ensures that saline is not administered when the vascular access device 404 may be disconnected or occluded.

In a similar manner, the first pump 102 may be stopped, causing an event message to be generated for the PCA pump 108. The reception of the event message causes the PCA pump 108 to generate an alarm indicating an interruption in the delivery of saline. In some instances, the PCA pump 108 is configured to generate an alert in response to receiving the stoppage event message from the first pump 102. Further, a reception of an occlusion alarm or other similar alarm from the first pump 102 may cause the PCA pump 108 to generate an alarm to cause an operator to pause a treatment.

As discussed above, the pump interconnectivity between the first pump 102 and the PCA pump 108 provides for a more coordinated delivery of saline and pain medication for a pain medication therapy. The hub device 106 enables the pumps 102/108 to communicate with each other without needing an external Ethernet or cellular network. Such a configuration ensures the pumps 102/108 are controlled simultaneously and prevents an error with one pump from going unnoticed during the delivery of the other pump.

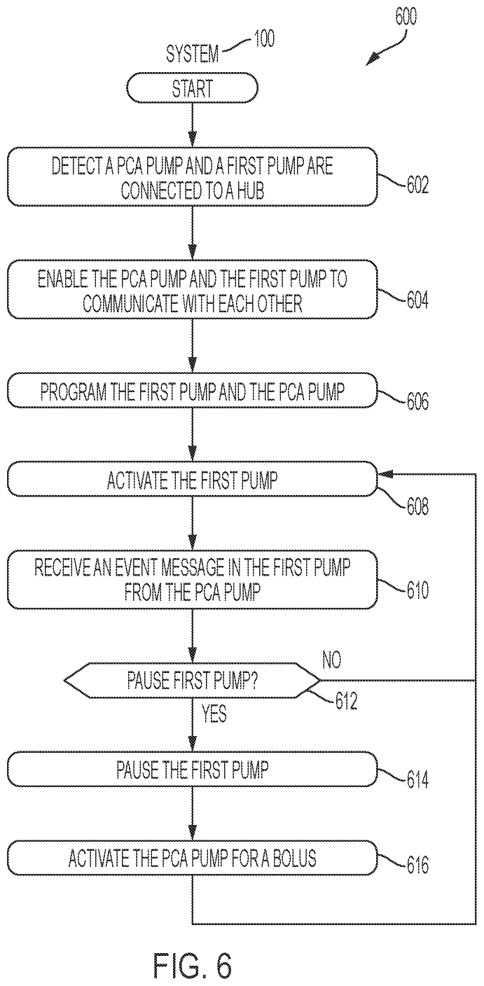

FIG. 6 is a flow diagram of an example procedure 600 for performing a pain medication therapy using the example hub device 106 disclosed herein. Although the procedure 600 is described with reference to the flow diagram illustrated in FIG. 6, it should be appreciated that many other methods of performing the steps associated with the procedure 600 may be used. For example, the order of many of the blocks may be changed, certain blocks may be combined with other blocks, and many of the blocks described may be optional. The operations described in the procedure 600 are specified by one or more instructions and may be performed among multiple devices including, for example, the first pump 102, the second pump 108, and/or the hub device 106.

The example procedure 600 begins when the hub device 106 detects a connection of the first pump 102 and the PCA (second) pump 108 (block 602). The hub device 106 may include connector pins that change voltage when the respective pumps 102/108 are physically connected. Further, the hub device 106 may establish communication with the pumps 102/108 using one or more communication protocols discussed herein. After communication is established, the hub device 106 enables the first pump 102 and the PCA pump 108 to communicate with each other directly or via broadcast messages (block 604).

After connection to the hub device 106, the PCA pump 108 and the first pump 102 are programed with parameters to perform respective treatments for the pain medication therapy (block 606). For example, the first pump 102 is programmed to provide a slow, but continuous dose of saline while the PCA pump 108 is programmed to provide a PCA bolus at a periodic rate. During the therapy, the first pump 102 is activated to provide saline (block 608). The saline may be provided first to wet the IV line and/or open a fluid connection to a patient's vein.

Next, the first pump 102 receives a message from the PCA pump 108 that indicates the PCA pump 108 is about to provide a bolus (block 610). In some embodiments, the message is transmitted in response to a scheduled bolus or a patient-initiated bolus. Further, in some embodiments, the message is transmitted as the PCA pump 108 begins to provide the bolus. The first pump 102 then determines if the infusion treatment is to be paused (block 612). When the first pump 102 is to be paused, the first pump 102 temporarily stops pumping to enable the PCA pump 108 to provide the bolus (blocks 614 and 616). After the bolus is complete, the first pump 102 resumes pumping (block 608). The first pump 102 may resume pumping after receiving an event message from the PCA pump 108 indicative that the bolus has been completed or after a default or specified time to provide the bolus. The example procedure 600 continues until the pain medication therapy. If one of the pumps 102/108 is removed or deactivated during the pain medication therapy, one or both pumps 102/108 may generate an alert. Further, when one of the pumps 102/108 detects an occlusion or leak, one or both pumps 102/108 may generate an alert and/or pause pumping.

### Conclusion

It will be appreciated that all of the disclosed methods and procedures described herein can be implemented using one or more computer programs or components. These components may be provided as a series of computer instructions on any conventional computer-readable medium, including RAM, ROM, flash memory, magnetic or optical disks, optical memory, or other storage media. The instructions may be configured to be executed by a processor, which when executing the series of computer instructions performs or facilitates the performance of all or part of the disclosed methods and procedures.

It should be understood that various changes and modifications to the example embodiments described herein will be apparent to those skilled in the art. Such changes and modifications can be made without departing from the spirit and scope of the present subject matter and without diminishing its intended advantages. It is therefore intended that such changes and modifications be covered by the appended claims.

It should be appreciated that 35 U.S.C. 112(f) or pre-AIA 35 U.S.C 112, paragraph 6 is not intended to be invoked unless the terms “means” or “step” are explicitly recited in the claims. Accordingly, the claims are not meant to be limited to the corresponding structure, material, or actions described in the specification or equivalents thereof.

1. A system for intravenously or epidurally administering pain medication, the system comprising:
a patient-controlled analgesia (“PCA”) pump configured to deliver a pain medication to a patient;
an infusion pump configured to deliver a fluid to the patient; and
a hub device including at least one pump stage with connectors that enable the PCA pump and the infusion pump to be removably connected to the hub device, the hub device including:
a communication interface configured to communicatively couple to each of the PCA pump and the infusion pump, and
a processor communicatively coupled to the communication interface, the processor configured to:
determine the PCA pump and the infusion pump are both communicatively connected to the communication interface,
enable the PCA pump and the infusion pump to communicate with each other for a pain medication therapy, and
enable the infusion pump to deliver the fluid to the patient when the infusion pump detects a period between periodic PCA pump boluses.

2. The system of claim 1, wherein the infusion pump is configured to detect the period between periodic PCA pump boluses using an event message received from the PCA pump, the event message indicative that the PCA pump is to administer or has administered a bolus of the pain medication.

3. The system of claim 1, wherein the PCA pump generates the event message after reaching a programmed scheduled bolus or receiving an input from a patient controller.

4. The system of claim 1, wherein the infusion pump is further configured to detect the period between periodic PCA pump boluses after receiving a bolus schedule that is indicative of times that the PCA pump is to administer a bolus of the pain medication.

5. The system of claim 1, wherein the communication interface is further configured to provide at least one of a controller area network (“CAN”) connection, an Ethernet connection, a serial connection, or a universal serial bus (“USB”) connection between the PCA pump and the infusion pump.

6. The system of claim 1, wherein the fluid is saline.

7. The system of claim 1, wherein the infusion pump is further configured to:
detect its removal from the hub device by detecting a lost connection with the communication interface; and
cause an alert to be generated that is indicative of a disconnection from the pain medication therapy.

8. The system of claim 1, wherein the infusion pump is further configured to:
detect a removal of the PCA pump from the hub device based on a lost connection with the communication interface or receive a message indicative that the PCA pump has been removed from the hub device; and
cause an alert to be generated that is indicative of a disconnection of the PCA pump.

9. The system of claim 1, wherein the PCA pump is further configured to:
detect its removal from the hub device by detecting a lost connection with the communication interface; and
cause an alert to be generated that is indicative of a disconnection from the pain medication therapy.

10. The system of claim 1, wherein the PCA pump is further configured to:
detect a removal of the infusion pump from the hub device based on a lost connection with the communication interface or receive a message indicative that the infusion pump has been removed from the hub device; and
cause an alert to be generated that is indicative of a disconnection of the infusion pump.

11. The system of claim 1, further comprising an intravenous (“IV”) y-connector with an outlet end connected to the patient, a first inlet end connected to the PCA pump, and a second inlet end connected to the infusion pump.

12. The system of claim 1, wherein the infusion pump includes at least one of a syringe pump, a linear peristaltic pump, a large volume pump (“LVP”), an ambulatory pump, or a multi-channel pump.

13. The system of claim 1, wherein the infusion pump is further configured to:
detect that the PCA pump has started the pain medication therapy; and
cause an alert to be generated that is indicative that the infusion pump has not yet been started for the pain medication therapy.

14. The system of claim 1, wherein the PCA pump is further configured to:
detect that the infusion pump has started the pain medication therapy; and
cause an alert to be generated that is indicative that the PCA pump has not yet been started for the pain medication therapy.

15. The system of claim 1, wherein the infusion pump is further configured to:
detect that a PCA pump has not yet been connected to the hub device; and
cause an alert to be generated that is indicative that the PCA pump should be connected to the hub device for the pain medication therapy.

16. The system of claim 1, wherein the PCA pump is further configured to:
detect that an infusion pump has not yet been connected to the hub device; and
cause an alert to be generated that is indicative that the infusion pump should be connected to the hub device for the pain medication therapy.

17. A hub apparatus configured to connect to (i) a patient-controlled analgesia (“PCA”) pump for delivering a pain medication to a patient, and (ii) an infusion pump for delivering a fluid to the patient, the hub apparatus comprising:
a pump stage with connectors that enable each of the PCA pump and the infusion pump to be removably connected to the hub apparatus;
a communication interface configured to communicatively couple to each of the PCA pump and the infusion pump; and
a processor communicatively coupled to the communication interface, the processor configured to:
determine the PCA pump and the infusion pump are both communicatively connected to the communication interface,
enable the PCA pump and the infusion pump to communicate with each other for a pain medication therapy, and
enable the infusion pump to deliver the fluid to the patient when the infusion pump detects a period between periodic PCA pump boluses.

18. The hub apparatus of claim 17, wherein the communication interface is configured to provide at least one of a controller area network (“CAN”) connection, an Ethernet connection, a serial connection, or a universal serial bus (“USB”) connection between the PCA pump and the infusion pump.

19. The hub apparatus of claim 17, further comprising a network connectivity stage configured to house the communication interface and the processor.

20. The hub apparatus of claim 19, wherein the pump stage is removably connected to the network connectivity stage and when connected, the pump stage is communicatively coupled to the network connectivity stage.
